(19) United States
(12) Patent Application Publication
(10) Pub. No.: US 2024/0090851 A1
(43) Pub. Date: Mar. 21, 2024

(54) SYSTEMS AND METHODS FOR ANESTHESIA DISPLAY

(71) Applicant: GE Precision Healthcare LLC, Wauwatosa, WI (US)

(72) Inventors: Mika Olli Kristian Särkelä, Uusimaa (FI); Helena Liisa Anneli Aito, Uusimaa (FI); Matti Juhani Laitinen, Uusimaa (FI); Milvi Kristiina Soosalu, Uusimaa (FI); Virpi Kristiina Lahdenmäki, Uusimaa (FI)

(21) Appl. No.: 17/932,434

(22) Filed: Sep. 15, 2022

(51) Int. Cl. A61B 5/00; G16H 20/17; G16H 40/67

(52) U.S. Cl. CPC A61B 5/743; A61B 5/7435; A61B 5/746; G16H 20/17; G16H 40/67

(57) ABSTRACT

Various methods and systems are provided for monitoring and displaying a patient status. In one example, the patient monitoring system includes a display device configured to display a patient status view include on or more multidimensional plots overlaying more than one of real time values, historical values, and upper and lower threshold values for a plurality of patient parameters, and additionally being configured to display an abbreviated representation of the one or more multidimensional plots that can be reached directly from the patient status view. The abbreviated representation is displayed in an unlaunched state, and displays a limited version of the respective multidimensional plot and is selectable to launch and enable the respective multidimensional plot to be seen.

### FIELD

Embodiments of the subject matter disclosed herein relate to a user interface for monitoring adequacy of anesthesia.

### BACKGROUND

During a surgery, a patient may be monitored for a plurality of parameters related to a physiological state of the patient, including a plurality of parameters related to adequacy of anesthesia (AoA). In this context, AoA refers to measurements that are typically used to monitor anesthetic drug effect on the patient. For example, patient central nervous system activity, such as electroencephalogram (EEG), can be used to monitor sedative drug effect. Another example are the parameters that have been demonstrated to follow analgesic drug level of the patient and in the case of inadequate analgesic dose reacting to nociceptive stimulus, these parameters may utilize one or several physiological measurements, such as EEG, electrodermal activity and hemodynamics.

Another category of parameters is related to patient circulatory state. For example, blood pressure, heart/pulse rate, cardiac output, and their various derivatives may be used for monitoring circulatory state of the patient. During the surgery, blood pressure of a patient may be monitored to ensure blood pressure is maintained within a target range. A patient with blood pressure dropping below the target range may be considered hypotensive. As a side effect, many anesthetic drugs affect to patient circulatory state. Therefore, during the surgery, it may be beneficial to the patient for a clinician (e.g., anesthesiologist) to consider multiple signals, each signal corresponding to one of the plurality of parameters, and changes over time in the more than one signal by observing the signals in a single display. This may allow the clinician to select an effective corrective action when at least one of the signals is outside of a threshold range.

### BRIEF DESCRIPTION

In one embodiment, a display device configured to display a patient status view including one or more multidimensional plots overlaying more than one of real time values of a plurality of patient parameters, historical values of the plurality of patient parameters, and lower and upper threshold values of the plurality of patient parameters, and additionally being configured to display an abbreviated representation of at least one of the one or more multidimensional plots that can be reached directly from the patient status view, wherein the abbreviated representation displays a limited version of the respective multidimensional plot, the abbreviated representation being selectable to launch and enable the respective multidimensional plot to be seen, and wherein the abbreviated representation is displayed while in an unlaunched state.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1A schematically shows a system for monitoring a patient during a surgery according to an embodiment.

FIG. 1B schematically shows an example of a display of patient monitoring system, according to an embodiment.

FIG. 2 shows a flow chart of an example of an overview method for displaying a plurality of patient parameters, according to an embodiment.

FIG. 3 shows a flow chart of an example of a method for using the plurality of patient parameters indicators, according to an embodiment.

FIG. 4 shows a flow chart of an example of method for automated treatment based on the methods of FIG. 2 and FIG. 3, according to an embodiment.

FIG. 5 shows graphs of raw adequacy of anesthesia (AoA) and circulatory indicators plotted over time, according to an embodiment.

FIG. 6 shows a first example of a multidimensional plot generated based on the raw AoA and circulatory indicators of FIG. 5, according to an embodiment.

FIG. 7 shows a first example of a display generated according to the method of FIG. 2.

FIG. 8 shows a second example of a display generated according to the method of FIG. 2.

FIG. 9 shows a third example of a display generated according to the method of FIG. 2.

### DETAILED DESCRIPTION

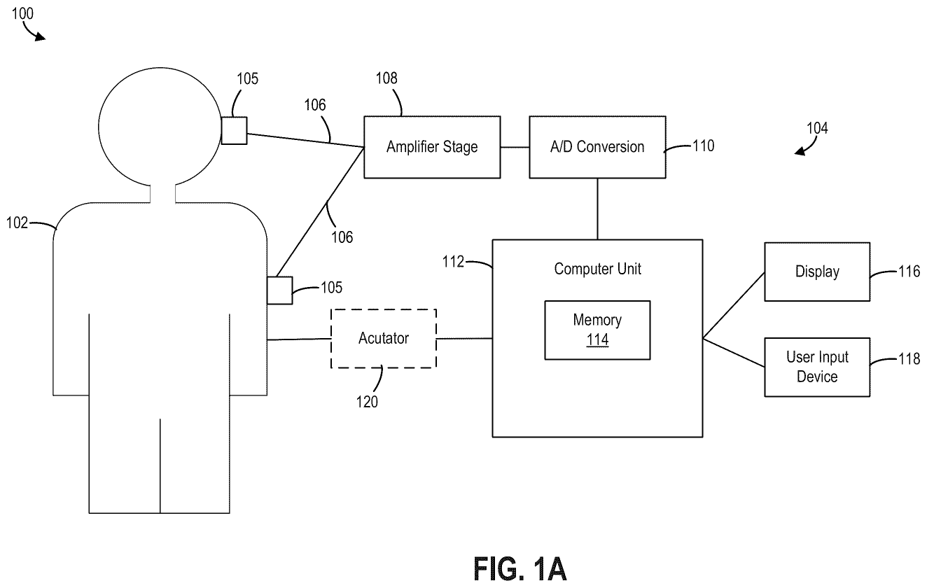

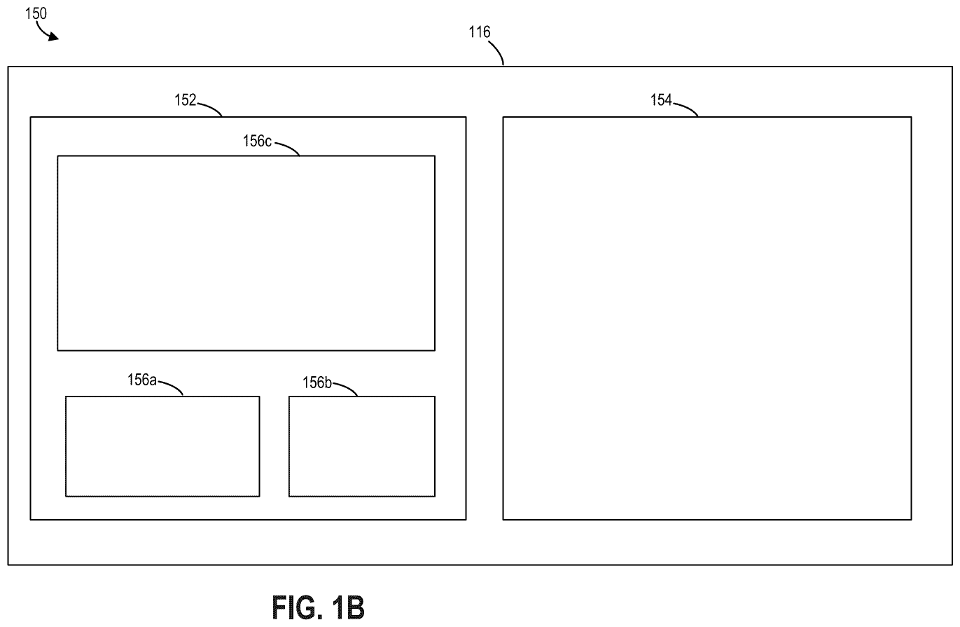

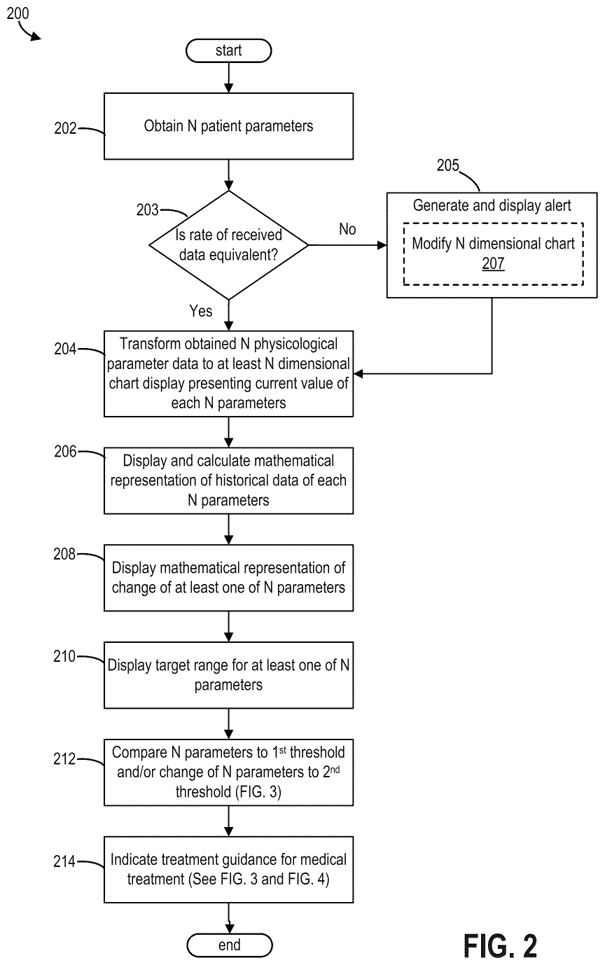

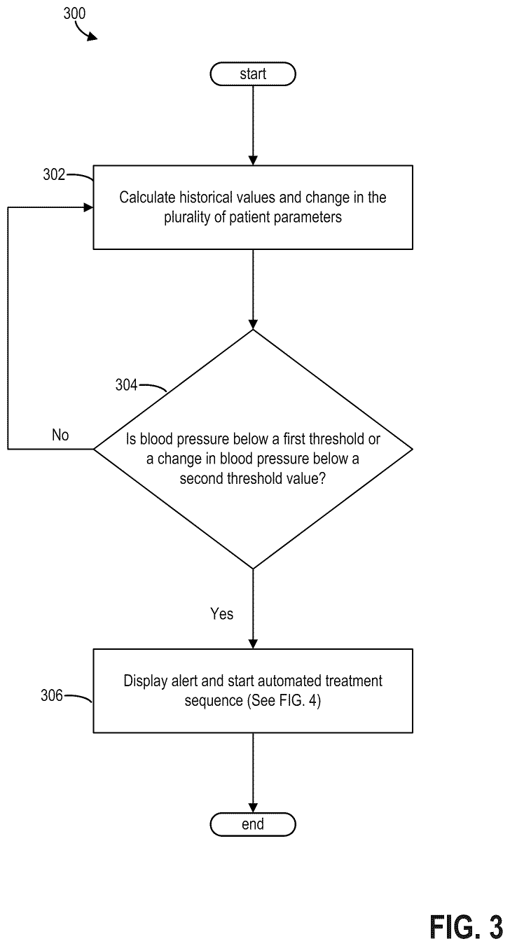

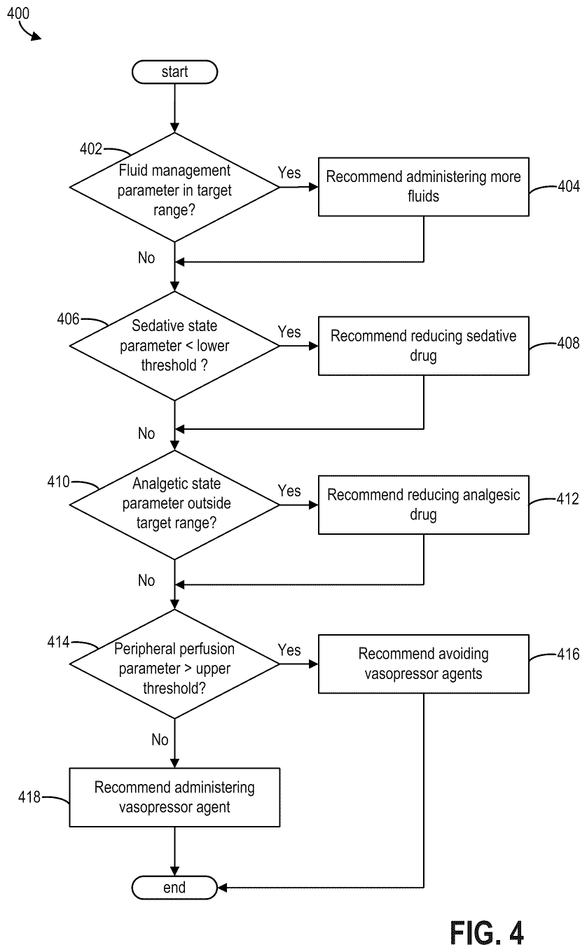

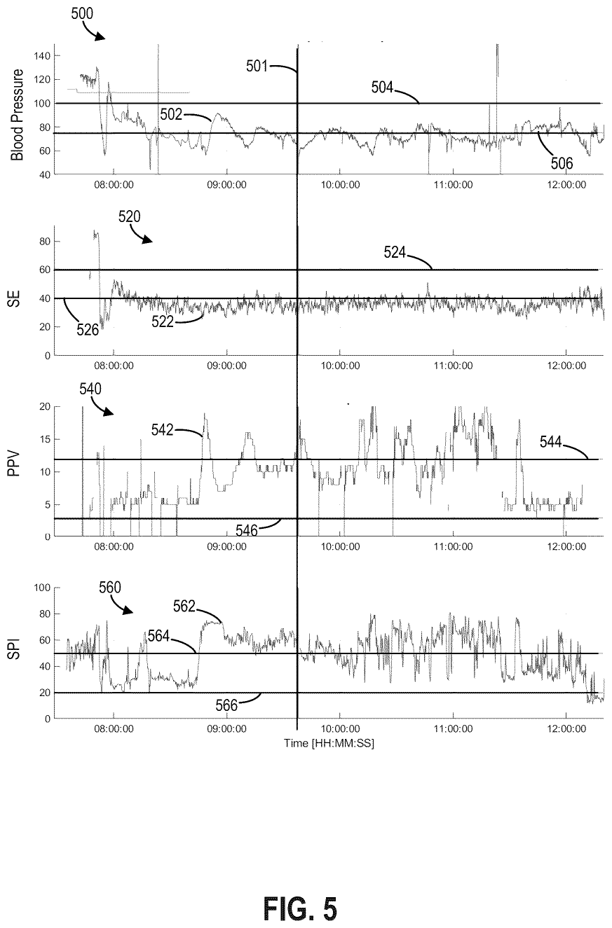

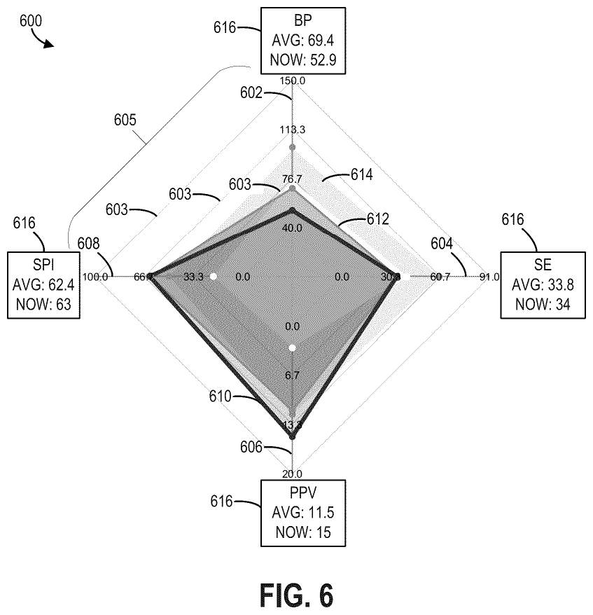

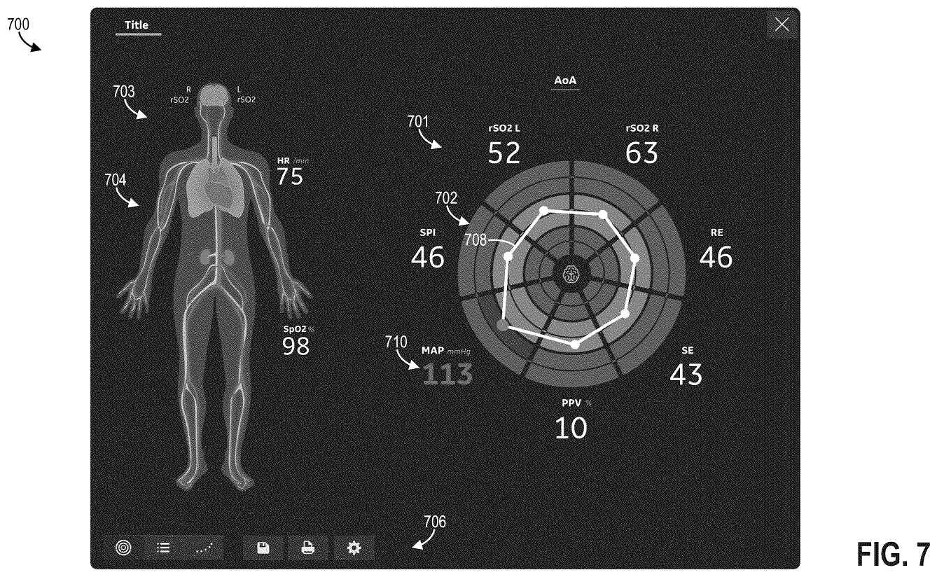

The following description relates to systems and methods for monitoring a patient during surgery and displaying a plurality of related patient parameters, such as a plurality of patient parameters related to adequacy of anesthesia (AoA) and circulatory state. An example of a system for monitoring a patient during surgery is shown in FIG. 1A. The system of FIG. 1A may output the plurality of patient parameters which may be displayed in a single multidimensional plot that a clinician may use to determine if any of the plurality of patient parameters are outside of a threshold range. An example of the display of FIG. 1A is shown schematically in FIG. 1B. The plurality of patient parameters may be output as raw data over time and converted via a method shown in FIG. 2 to a multidimensional plot. Additionally, the system may alert a clinician when a patient parameter is outside of a threshold range. For example, the clinician may be alerted when a blood pressure of the patient drops below a threshold value indicative of hypotension. In response to a drop in blood pressure below a threshold value, the system may output treatment recommendations on the display for patient care. The treatment recommendations may be determined based on the monitoring of the plurality of patient parameters, as shown in FIGS. 3 and 4. In some examples, the treatment recommendations may be automatically provided by a controller of the system. Such automated treatment recommendations may enable a practitioner to select a suitable and effective course of action. An example of raw data which may be utilized in the method of FIG. 2, is shown in FIG. 5. An example of a multidimensional plot which may be generated according to the method of FIG. 2, based on the raw data of FIGS. 5, is shown in FIG. 6. The multidimensional plot may be presented at a user display. Embodiments of the user display, depicting at least an example of a multidimensional plot, are shown in FIGS. 7, 8, and 9.

Turning now to FIG. 1A a schematic 100 is shown of a patient 102 interfacing with a patient monitoring system 104. Patient monitoring system 104 may be used to monitor in real-time a condition of patient 102. Further, patient monitoring system 104 may be used during a surgical procedure to determine and display a physiological state of the patient including values related to AoA and circulatory state among others.

Physiological signals 106 may be collected by monitoring devices 105 coupled to the patient. In one example monitoring devices 105 may include electroencephalography (EEG) electrodes configured to measure electrical activity of the brain. Additionally, monitoring devices 105 may include a pulse oximeter configured to pass light through a finger of the patient and measure oxygen saturation of the patient's blood. Further, monitoring devices 105 may additionally include an invasive blood pressure monitor configured to continuously measure the patient's blood pressure.

Physiological signals 106 may be communicated from the monitoring devices 105 to an amplifier stage 108. Amplifier stage 108 may amplify physiological signals 106 and communicate the amplified physiological signals 106 to an analog/digital (A/D) converter 110. A/D converter 110 may convert the amplified analog physiological signals to digital signals which may be input into computer unit 112.

Computer unit 112 may include one or more processors configured to execute instructions stored in non-volatile memory 114. As one example, computer unit 112 may include instructions for converting amplified digital physiological signals received from A/D converter 110 into a plurality of patient parameters. The plurality of patient parameters may include values related to AoA and circulatory state which may be computed from physiological data gathered from monitoring devices 105 configured to interface with patient 102. As one example, an output of the EEG electrodes may be monitored and processed by instructions included in non-volatile memory 114 to determine a state entropy of a patient. State entropy (SE) may be a numerical integer between 0 and 91 indicative of an irregularity of an EEG output. SE may be used to determine a sedative effect of an anesthetic drug. For example, an SE value below 60 may be considered adequate for commencement of a surgical procedure. As another example, the pulse oximeter output may be processed by instructions included in non-volatile memory 114 to determine a surgical pleth index (SPI) of a patient. An SPI value may determine an analgesic effect and nociceptive response experienced by the patient. Further a change in SPI over time may be indicative of changes in peripheral blood perfusion. Additionally, pleth variability index (PVI) may be determined by analyzing plethysmograph waveform signal, which may be obtained from oximeter output. PVI may be indicative of a euvolemia and/or fluid responsiveness of the patient. The plurality of patient parameters may additionally include blood pressure as measured by signals generated by the invasive blood pressure monitor.

A display 116 and user-input device 118 may be operably coupled to computer unit 112. In one example user-input device 118 may include peripheral devices such as a keyboard and/or mouse. In other examples, user-input device 118 may be a touch sensitive screen of a display 116. Display 116 may be configured to display a patient status view including the plurality of patient parameters, such as the plurality of patient parameters related to AoA and circulatory state, such as SE, SPI, PVI, and blood pressure. Due to size constraints of display 116 and a demand to display multiple different patient parameters, it may be advantageous to display related patient parameters as information dense graphics. For example, the related plurality of patient parameters may be displayed as a single multidimensional plot.

One embodiment of a patient status view is shown by schematic 150 in FIG. 1B. The schematic 150 may be shown on display 116 and may include multiple areas. Schematic 150 shows a first area 152 and a second area 154. More than two areas may be considered, however, within a scope of the present disclosure. The first and second areas may be similar or dissimilar in size. In one example, first area 152 may include multidimensional plots of related patient parameters, such as the plurality of patient parameters relevant to AoA. Further, second area 154 may display additional patient parameters also related to the current state of the patient including temperature, heart rate, and blood oxygen levels among others.

First area 152 may include first sub-area 156a, second sub-area 156b, and third sub-area 156c (collectively referred to as sub-areas 156). Sub-areas 156 may be of equal or different areas and may be located at different locations within first area 152. As one example first sub-area 156a may include a text box including numbers corresponding to patient parameters relevant to AoA. Second sub-area 156b may include a first multidimensional plot of a related plurality of patient parameters. Third sub-area 156c may include a second multidimensional plot of a plurality of patient parameters, different from the plurality of patient parameters shown in second sub-area 156b. First area 152 and second area 154 may each include greater or fewer sub-areas without departing from the scope of the disclosure. Embodiments of display 116 may be discussed further below with respect to FIGS. 7-9.

Returning to FIG. 1A, patient monitoring system 104 may optionally include an actuator 120 coupled to computer unit 112. Actuator 120 may be coupled to patient 102 and may be configured to deliver treatments to patient 102 in response to an output of computer unit 112. Alternatively, computer unit 112 may output treatment recommendations regarding a treatment of patient 102 which may be displayed on display 116 and may be carried out by the clinician.

In an event of a patient parameter increasing or decreasing outside of a threshold range during a surgery, remedial treatment of the patient may be demanded. An effective treatment may be chosen by considering a plurality of patient parameters concurrently, rather than monitoring individual patient parameters. For example, a drop in patient blood pressure below a threshold may be considered a hypotensive event. The hypotensive event during a surgery may be treated by fluid therapy, administration of vasopressors, reducing a dosage of sedative or analgesic drugs, or combinations thereof. An effective treatment may depend on which patient parameter, or plurality of patient parameters are outside of the threshold range. It may be considered beneficial for a practitioner to view one or more of the plurality of patient parameters in a single plot along with patient blood pressure so that patient parameters outside of the threshold range may be identified without demanding navigation between views on a display. Further trends (e.g., increase or decrease over time) of the plurality of patient parameters over time may be easily identified and used to inform a treatment selection. Considering SE, SPI, change in SPI, and PVI together in the event of a patient blood pressure decreasing below a threshold (e.g., hypotension) in combination with an automated treatment recommendation may lead to a clinician making a quicker and more effective decision regarding a treatment to correct the hypotension thus minimizing an amount of time the patient is hypotensive. Minimizing an amount of time that an anesthetized patient is hypotensive may result in more favorable post-surgical outcomes for the patient.

Turning now to FIG. 2, a method 200 is shown for displaying a plurality of patient parameters. The display may show the plurality of patient parameters in such a way that the plurality of patient parameters may be observed concurrently on a compact display and may aid a clinician in making expedient patient care decisions when a patient parameter varies outside a threshold value. Method 200 may be particularly advantageous when employed while the patient is under anesthesia. Method 200 may be executed using executable instructions stored in the non-transitory memory of a computer unit, such as computer unit 112 of FIG. 1A.

At 202, method 200 includes obtaining N patient parameters. The N patient parameters may be the plurality of patient parameters collected by a patient monitoring system such as patient monitoring system 104 of FIG. 1A. The patient monitoring system may collect physiological signals from monitoring devices and convert the signals to patient parameters. The N patient parameters may be related to assessing a status of the patient, such as AoA, hemodynamics, and/or fluid balance. In one embodiment, the N patient parameters may include blood pressure and/or the plurality of patient parameters relevant to AoA such as SE, PVI, SPI. In one example N may be equal to four. However, N patient parameters may be greater than or less than four without departing from the scope of this disclosure. The obtained N patient parameters may be stored in the non-transitory memory of the computer unit.

At 203, method 200 includes determining if rates of received for each of the N patient parameters at 202 are equivalent. As one example SPI, blood pressure, SE, and PVI may all be obtained at a rate of one measurement per second. In one example, determining if rates of data received for each of the N patient parameters are equivalent may include determining if one or more of the N patient parameters received at 202 has been updated within a time period. In one example, the time period may be 1 minute. If data for one or more of the N patient parameters is received at a non-equivalent rate, method 200 proceeds to 205 and includes generating and displaying an alert. An alert may include a written message. In some embodiments, the alert may include at 207 modifying an N dimensional plot displaying the N patient parameters, the N dimensional plot described further below with respect to step 204. Modifying the N dimensional plot may include altering a color or shading related to the patient parameter that is received at a non-equivalent rate. Additionally or alternatively, the patient parameter received at the non-equivalent rate may not be included in the N dimensional plot. In this way the clinician may be alerted to a patient parameter which is not updated at a rate not equivalent to the remaining N patient parameters. Method 200 proceeds to 204.

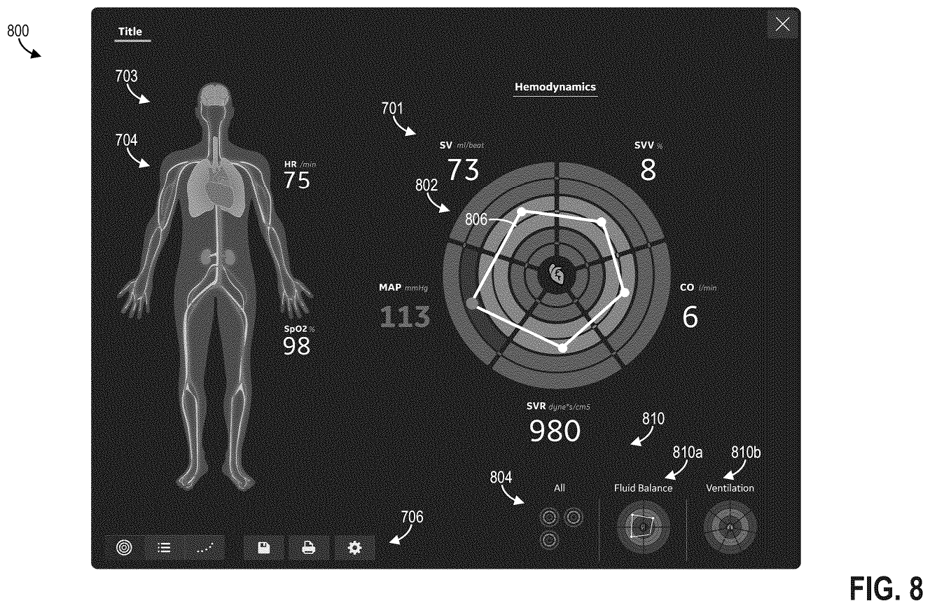

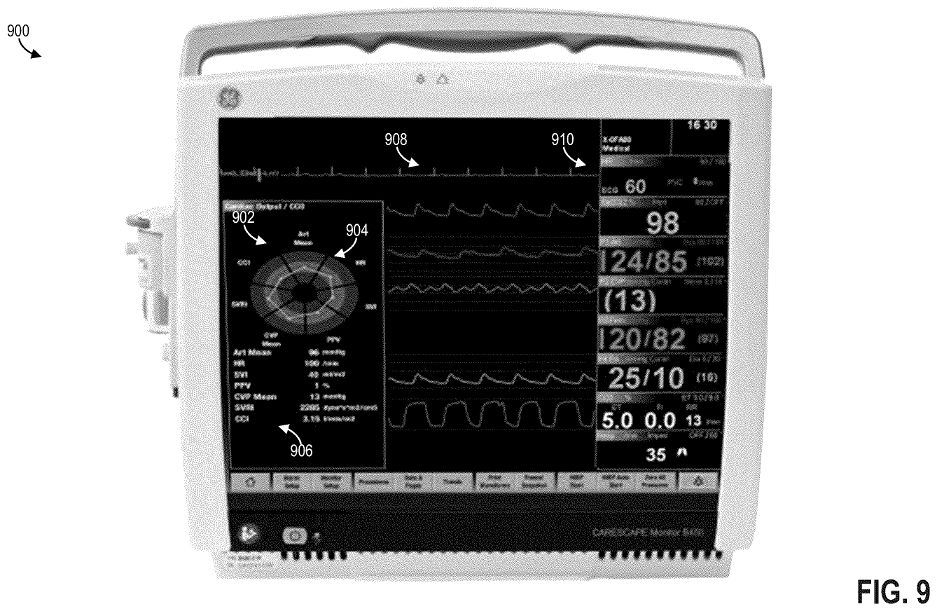

If the rates of data received at 202 are equivalent, method 200 proceeds to 204 and includes transforming the N patient parameters into a N dimensional chart presenting a real time value of each of the N patient parameters simultaneously. As one example, the N dimensional chart may be a geometrically-shaped multidimensional plot such as the polygon multidimensional plot shown in FIG. 6 below or the circular multidimensional plots shown in FIGS. 8-9. In other examples, the N dimensional chart may a be a different type of graph such as a clustered bar graph, or other graphs showing multidimensional data. The N dimensional chart displaying all N parameters conserves space on a display of the patient monitoring system (such as display 116 of FIG. 1A) and allows a practitioner to view all of the N patient parameters in a single area or sub-area of the display. For example, the N dimensional chart may be displayed in one of sub-areas 156 of FIG. 1B.

At 206, method 200 includes calculating and overlaying a representation of historical data of each of the N patient parameters on the N dimensional chart. The historical data may be automatically calculated by using the stored values of the plurality of patient parameters in the memory of the computing unit and averaging at least two values of an N-patient parameter obtained immediately prior to obtaining the real time value. In this way, the historical data may be updated at a rate equivalent to the rate at which real time values are updated. In one example, a number of prior patient parameter values included in the historical data average may include patient parameters value obtained over 10 minutes immediately prior to obtaining the real time value. In another example the historical data average may include patient parameters collected for a duration between 2 minutes and 20 minutes immediately prior to obtaining the real time value. Displaying historical data of the N patient parameters on the N dimensional chart may allow the clinician to quickly observe trends in the N patient parameters (e.g., increasing or decreasing) without relying on the clinician's memory, and without separately recording a historical value or demanding user input to access and navigate to historical data from the computing unit.

At 208, method 200 includes displaying a representation of a change of at least N patient parameters on the N dimensional chart. The change may be a numerical difference between the real time value obtained at step 204 and the corresponding historical value calculated at step 206. In some examples, a rate of change may also be calculated and displayed. The change may be displayed for any or all of the N patient parameters. Displaying the change may include adding an additional dimension to the N dimensional chart for each changed value to be displayed. In other examples a direction in or magnitude of change may be displayed symbolically on the N dimensional chart. For example, change may be indicated by using up or down arrows of different sizes. In one example change in SPI may be displayed.

At 210, method 200 includes displaying a target range for at least one of the N patient parameters and/or change in N patient parameters on the N dimensional chart. A target range may bound by an upper and/or lower threshold a value for the N patient parameter. In one example the target range may correspond to an age and weight of the patient and may be stored in a look up table in the computing unit. In some examples the target range may be overlaid with the real time and historical values of the N patient parameters on the N dimensional chart.

At 212, method 200 includes indicating if one of the N patient parameters is outside of a first threshold and/or the change of the N patient parameters is outside a second threshold. The first threshold may be the target range bounded by an upper and lower threshold as described above. The second threshold may be an upper threshold of an amount of change in one or more of the N patient parameters. In one example, indicating the N patient parameter outside the target range may include a visual indication such as showing the N patient parameter in a modified color on the N dimensional chart. In an alternate example, indicating the N patient parameter outside the target range may include a separate written notification appearing on a separate sub-area of the display showing the N dimensional chart. As a further example, N patient parameters outside of the target range may be indicated audibly via an alarm and/or beep. An example of comparing one of the N patient parameters to the target range is discussed further below with respect to FIG. 3.

At 214, method 200 includes indicating treatment guidance based at least partially on alerts indicated at 212. Treatment guidance may be generated automatically in real-time by instructions included on the computer unit configured to suggest a treatment by inputting each of the N patient parameters. An example of a method for generating treatment guidance is discussed further below with respect to FIG. 4. In some examples, the treatment indicated by the treatment guidance may be automatically administered to the patient by an actuator such as actuator 120 of FIG. 1A. In this way, a treatment may be delivered by the practitioner faster than if the clinician was just considering each of the N patient parameters displayed individually. Further, the treatment may be more likely to be effective because each of the real time N patient parameters and their historical values have been considered. An effective treatment applied quickly may result in more favorable patient outcomes.

Turning now to FIG. 3, a method 300 for monitoring a patient under anesthesia is shown. Method 300 may include an embodiment of method 200 wherein the N patient parameters are the plurality of patient parameters relevant to AoA and circulatory state, and indicating a treatment guidance is responsive to a patient experiencing hypotension. Method 300 may be executed using executable instructions stored in the non-transitory memory of a computer unit, such as computer unit 112 of FIG. 1A.

At 302, method 300 includes calculating historical values and a change in the plurality of patient parameters. The plurality of patient parameters may be the patient parameter relevant to AoA (e.g., SE and SPI) and circulatory state (e.g., PVI and blood pressure). Historical values may be averaged as described above with respect to 206 of method 200. In one example, historical values of each of the plurality of patient parameters may be calculated. The change in one or more of the plurality of patient parameters may be calculated. For example, a change in SPI may be a numerical difference between a historical SPI value and the current SPI value as described above with respect to 208 of method 200. A change in blood pressure may also be calculated as described above with respect to 208 of method 200. The historical values and changes in the plurality of patient parameters may be displayed on a multidimensional plot as described as described above with respect to method 200.

The 4 dimensional plot may include information relevant for the clinician to make a treatment decision if the patient becomes hypotensive. At 304, method 300 includes determining if the blood pressure is below a first threshold for a change in blood pressure is above a second threshold. The first threshold value may be a value, below which the patient would be considered hypotensive. The second threshold may be a threshold change in blood pressure, above which may indicate an onset of a hypotensive event. In one example, the first threshold may be 90 mm Hg systolic or 60 mm Hg diastolic. If the blood pressure is above the first threshold value and the change in blood pressure is below the second threshold value, method 300 returns to 302. If the blood pressure is below the first threshold value or the change in blood pressure is above the second threshold, method 300 proceeds to 306 and includes displaying an alert and starting an automated treatment sequence. As examples, the displayed alert may be a written alert or a color based code, notifying the practitioner that the patient is undergoing a hypotensive event. The automated treatment sequence may be generated by instructions for making treatment recommendations based on the plurality of patient parameters (e.g., blood pressure, SPI, PVI, and SE) displayed on the 4 dimensional plot. The instructions may be described further below with respect to FIG. 4.

Turning now to FIG. 4, a method 400 is shown for recommending treatments for a hypotensive patient under anesthesia during a surgical procedure. Method 400 may rely on a plurality of patient parameters as inputs which may be obtained as described above with respect to FIG. 3. Method 400 may be executed using computer executable instructions stored in the non-transitory memory of a computer unit, such as computer unit 112 of FIG. 1A. In some examples, the treatment recommendations of method 400 may be automatically administered to the patient via instructions sent from the computer unit to an actuator such as actuator 120 of FIG. 1A.

At 402, method 400 includes determining if a fluid management parameter is within a target range. The fluid management parameter may include PVI, PPV (pulse pressure variation), SPV (systolic pressure variation), SVV (stroke volume variation), or change in PI (peripheral perfusion index). The target range may include an upper threshold and/or a lower threshold bound. As one example, the target range for PVI may include an upper threshold and if the PVI is greater than or equal to the upper threshold, the patient is considered responsive to fluid administration. The upper threshold may be included in a target range displayed on the multi-dimensional plot described above with respect to FIG. 2. If the fluid management parameter is outside of the target range (e.g., PVI is above the upper threshold value) the patient may be considered responsive to fluids and method 400 continues to 404 and includes recommending administering more fluids. In one example, the recommendation may be conveyed to the clinician by a written or symbolic message on a display such as display 116 of FIG. 1A. Additionally or alternatively, the recommendation may be conveyed as an audio message.

If the fluid management parameter is not outside the target range, method 400 continues to 406. Method 400 also proceeds to 406 following 404. At 406, method 400 includes determining if a parameter for sedative state is below a lower threshold value. A sedative state parameter may include SE, BIS (bispectral index scale) or PSI (patient state index). The threshold value may be the same value lower threshold value included in a target range displayed on the multi-dimensional plot described above with respect to FIG. 2. If the sedative state parameter (e.g., SE) is less than the threshold value, method 400 continues to 408 and includes recommending reducing an amount of sedative drug being administered to the patient. In one example, the recommendation may be conveyed to the clinician by a written or symbolic message on the display. Additionally or alternatively, the recommendation may be conveyed as an audio message.

If the sedative state parameter is not lower than the threshold value, method 400 proceeds to 410. Method 400 also proceeds to 410 following 408. At 410, method 400 includes determining if an analgetic state parameter is outside a target rage. The analgetic state parameter may include SPI, NOL (nociceptive level), or ANI (analgesia nociception index). As one example, a target range for SPI may include a lower threshold and SPI values may be indicative of excess analgesic drug. The target range may be the same range displayed on the multi-dimensional plot described above respect to FIG. 2. If the analgesic state parameter is outside the target range, (e.g., SPI is less than the lower threshold value), method 400 continues to 412 and includes recommending reducing an amount of analgesic drug. In one example, the recommendation may be conveyed to the clinician by a written or symbolic message on the display. Additionally or alternatively, the recommendation may be conveyed as an audio message.

If the analgesic state parameter is within the target range, method 400 proceeds to 414. Method 400 also proceeds to 414 following 412. At 414, method 400 includes determining if a peripheral perfusion parameter is greater than an upper threshold. In one example, the peripheral perfusion parameter may include change in SPI. The change in SPI may be determined by comparing a current SPI value to historical values as described above with respect to FIGS. 2 and 3. In some examples, the change in SPI may also be displayed on the multi-dimensional plot as described above with respect to FIG. 2. In alternate examples, the peripheral perfusion parameter may include peripheral perfusion index (PI) or stroke volume variation (SVR). If the peripheral perfusion parameter is greater than the upper threshold value, method 400 proceeds to 416 and includes recommending avoiding vasopressor agents. In one example, the recommendation may be conveyed to the clinician by a written or symbolic message on the display. Additionally or alternatively, the recommendation may be conveyed as an audio message. Method 400 ends.

If the peripheral perfusion parameter is not greater than the upper threshold value, method 400 proceeds to 418. At 418, method 400 includes recommending administering a vasopressor agent. The recommendation may be conveyed to the clinician by a written or symbolic message on the display. Additionally or alternatively, the recommendation may be conveyed as an audio message. Method 400 ends.

In this way, recommendations provided via method 400 may aid the clinician in determining a treatment or combination of treatments for a hypotensive patient. Automatic recommendations provided in real-time based on the values displayed to the clinician may minimize an amount of time the patient spends in a hypotensive state while sedated during surgery, thereby improving an outcome for the patient post-surgery.

Methods described in FIGS. 2-4 above may cover obtaining a plurality of patient parameters and transforming the obtained data into a useful multidimensional display and providing recommendations for treatment in response to a patient becoming hypotensive. The plurality of patient parameters may be collected as raw data and stored along with corresponding time points in a memory of a computer unit, such as computer unit 112 of FIG. 1A. Examples of raw reports which may be used in the methods of FIGS. 2-4 are depicted in FIG. 5, and examples of a multidimensional plots, which may be generated according to method 200, are shown in FIGS. 6-9.

Turning to FIG. 5, it shows four plots 500, 520, 540 and 560. Each plot shows a patient parameter on a y-axis as a function of time on an x-axis. The x-axis of each plot may be the same x-axis, thereby showing concurrent collection of data at plots 500, 520, 540, and 560. The patient parameters may be collected by a patient monitoring system such as patient monitoring system 104 of FIG. 1A. The plots of FIG. 5 may correspond to data stored within a non-volatile memory of a computer unit of the patient monitoring system, but may not be output to a display of the patient monitoring system.

Plot 500 is a plot of patient blood pressure as function of time. Trace 502 corresponds to measured blood pressure of a patient and may be obtained from an invasive blood pressure device coupled to the patient and converted to blood pressure by the computing unit of the patient monitoring system. Line 504 corresponds to an upper threshold blood pressure value and line 506 corresponds to a lower threshold blood pressure value. When trace 502 is below line 506, a hypotensive event may be indicated.

Plot 520 is a plot of SE as a function of time. Trace 522 corresponds to a measured SE of a patient and may be obtained from an EEG monitor coupled to the patient and converted to SE by the computer unit. Line 526 corresponds to a lower threshold entropy value and line 524 corresponds to an upper threshold entropy value.

Plot 540 is a plot of patient PPV as a function of time. Trace 542 corresponds to measured PPV of the patient and may be obtained from the invasive blood pressure monitor coupled to the patient. A signal from the invasive blood pressure monitor may be converted to PPV by the computer unit of the patient monitoring system. Line 544 corresponds to an upper threshold PPV value and line 546 corresponds to a lower PPV threshold value.

Plot 560 is a plot of SPI as a function of time. Trace 562 corresponds to measured SPI of the patient and may be obtained from the pulse oximeter coupled to the patient. The signal from the pulse oximeter may be converted to SPI in addition to PPV, by the computer unit. Line 564 corresponds to an upper threshold SPI value and line 566 corresponds to a lower threshold SPI value.

The information provided by plots 500, 520, 540, and 560 of FIG. 5, for any given time, may be displayed in a multidimensional plot. The multidimensional plot may be updated at a desired frequency while the patient is being monitored. As one example, a time corresponding to a hypotensive event may be displayed in a N dimensional chart as described above with respect to FIG. 2. An example of an embodiment of such a N dimensional chart is illustrated as a polygon multidimensional plot 600, also referred to as a spider graph 600, as shown in FIG. 6. Spider graph 600 may include radial axes for each of the plurality of patient parameters of FIG. 5. Radial axes may include a first axis 602 showing blood pressure, a second axis 604 showing entropy, a third axis 606 showing PPV, and a fourth axis 608 showing SPI. Spider graph 600 may also include circumferential axes 603 intersecting the radial axes according to numerical scales on the radial axes. The radial and circumferential axes may define a plurality of sections of spider graph 600, such as section 605 shown in FIG. 6.

Trace 610 corresponds to real time values of the patient parameters plotted in spider graph 600. Values shown by trace 610 correspond to values from traces 502, 522, 542, and 562 at the time indicated by line 501 of FIG. 5. Trace 612 corresponds to historical values of the patient parameters. The historical values may be calculated by taking an average of the values of the patient parameters over a duration immediately preceding the time of the real time values. As one example, trace 612 may be calculated by averaging values of the traces of FIG. 5 for 10 minute prior to line 501. In one example, one or more sections or parts of sections of spider graph 600 within areas defined by traces 610 and 612 may be shaded.

Spider graph 600 may also show lower and upper threshold values of the plurality of patient parameters. As one example, one or more sections and/or portions of sections of spider graph 600 may be shaded to indicate a target range bound by lower and upper threshold values of the plurality of patient parameters as shown by area 614. Spider graph 600 may also include text boxes 616. Text boxes may include numbers corresponding to real time and/or historical values for the plurality of patient parameters. In one example, a text box for each of the plurality of patient parameters may be located next to the corresponding radial axis. In an alternate example, a single text box including numbers corresponding to all of the plurality of patient parameters may be located in a separate area or sub-area of a patient status view.

A practitioner looking at spider graph 600 may determine that the patient's PPV is higher than the historical average and higher than the upper threshold value. Further, spider graph 600 also shows the patient's SE is the same as the historical average but is lower than the lower threshold value. Additionally, spider graph 600 shows the patient's SPI is the same as the historical average but is higher than the upper threshold value. The computer unit of the patient monitoring system may also be configured to recommend a treatment for the patient based on the patient parameters displayed in spider graph 600 according to method 400 of FIG. 4. In the examples shown in FIGS. 5 and 6, fluid administration and a reduction of sedative drugs may be recommended based on the PPV being above the upper threshold and the SE below the lower threshold. Further, administration of a vasopressor agent may be recommended because a change in SPI is less than a threshold indicated at 414 of FIG. 4. In this way an appropriate treatment for the hypotensive event may be determined by the clinician by observing multiple patient parameters at a single display area, in an overlaid manner showing threshold values and/or historical values and/or changes in values. Further, the appropriate treatment may be automatically recommended, allowing the clinician to quickly address the hypotensive event.

A shaped multidimensional plot of a plurality of patient parameters (such a spider graph 600) may be incorporated into a patient status view that may be displayed on a display device such as display 116 of FIG. 1A. A first example of a patient status view 700 is shown in FIG. 7. Patient status view 700 may be viewed by a clinician (e.g., anesthesiologist) during a surgery while a patient is under anesthesia. Patient status view 700 may include multiple areas (such as first area 152 and second area 154 of FIG. 1B) showing patient parameters relevant to a physiological status of the patient. Patient status view 700 may include shaped multidimensional plot 702 positioned in first area 701. Shaped multidimensional plot 702 may be a circular multidimensional plot and may be referred to as spider graph 702. A patient diagram 704 may occupy a second area 703 of patient status view 700, the second area 703 not overlapping with, e.g., spaced away from, the first area 701. Patient status view 700 may also include navigation buttons 706. Navigation buttons 706 may be positioned along a bottom of patient status 700 in second area 703, and not overlapping spider graph 702 or patient diagram 704. In one example spider graph 702 may occupy less than 100% of an area of patient status view 700.

Spider graph 702 may be a 7-dimensional graph showing 7 patient parameters relevant to AoA and circulatory state. Spider graph 702 may include trace radial and circumferential axes defining sections of spider graph 702 as described above with respect to FIG. 6. In one example, spider graph 702 may include trace 708 showing real time values of the plurality of patient parameters according to axes of spider graph 702. Sections of spider graph 702 may be shaded corresponding to upper and lower threshold values so that sections corresponding to patient parameter values within a target range are shaded differently than those that are outside the target range. Different shading may alternatively or additionally be displayed as different colors, and/or different brightness/saturations of the colors. In one example, axes of spider graph 702 may be scaled such that the area between respective threshold values for each parameter is equal across all axes of the spider plot. Although not shown in spider graph 702, spider graph 702 may also display historical values of the 7 patient parameters as second trace overlaid on trace 708. Spider graph 702 may also include text boxes 710 including numerical values corresponding to real time values shown by trace 708. In some examples, a section and/or text box corresponding to a real time value outside of the upper or lower threshold values may be shaded and/or colored differently as part of a notification.

A second example of a patient status view 800 according to an embodiment is shown in FIG. Patient status view 800 may include elements of patient status 700 which will not be renumbered or reintroduced. Patient status 800 may include a shaped multidimensional plot 802. Shaped multidimensional plot 802 may be a circular multidimensional plot and may be referred to as spider graph 802. Spider graph 802 may be a 5 dimensional plot showing a plurality of patient parameters relevant to hemodynamics which may be different from a plurality of patient parameters relevant to AoA. Similar to spider graph 702, spider graph 802 may include trace 806 corresponding to real time values of the patient parameters. Another trace (not shown) may be included on spider graph 802, corresponding to historical values of the plurality patient parameters. Spider graph 802 may also include sections defined by circumferential and radial axes as described above with respect to FIG. 6. Sections of spider graph 802 may be shaded and/or colored to indicate a range bounded by upper and lower threshold values for each of the plurality of patient parameters.

Patient status view 800 may also include a spider graph menu 804. Spider graph menu 804 may be positioned along a bottom edge of patient status view 800 within first area 701 and may be spaced away from other displayed features of patient status 800. Spider graph menu 804 may include abbreviated representations of shaped multidimensional plots including a first abbreviated representation 810a and a second abbreviated representations 810b (collectively referred to as abbreviated representations 810) which may be reached from patient status view 800. Abbreviated representations 810 may be limited versions of spider graphs shown such as spider graph 802 and 702, and may therefore include fewer elements than spider graph 802. As one example, abbreviated representations 810 may not include text boxes. As another example, abbreviated representations may not include a trace corresponding to real time values of the patient parameters as shown in second abbreviated representation 810b. In an example where the abbreviated representation includes a trace corresponding to real time values of the patient parameters, as shown in the first abbreviated representation 810a, the trace may be updated in real time.

Abbreviated representations shown on spider graph menu 804 may be selectable. Selecting an abbreviated representation from spider graph menu 804 may allow a clinician to launch an abbreviated representation on patient status view 800. Abbreviated representations 810 shown in spider graph menu 804 may be considered to be in an unlaunched state. Launching an abbreviated representation may include showing the corresponding multidimensional plot in first area 701 of patient status view 800 so that the multidimensional plot may be seen by the clinician. Each spider graph may display a different plurality of patient parameters relevant to different aspects of patient care. If a respective patient parameter displayed on abbreviated representations 810 is outside a threshold range, a notification may be generated. In one example the notification may include automatically launching the abbreviated representation including the patient parameters which is outside the threshold range. In some examples spider graph menu 804 may also allow the practitioner to choose to display one or more shaped multidimensional plots at once. Shaped multidimensional plots such as spider graphs may plot multiple variables in a space efficient manner. In this way an amount of information viewed at a single screen without further navigation may be increased. The shaped multidimensional plot may be small enough that multiple shaped multidimensional plots may be displayed at once, while still being readable and useful to the clinician.

A shaped multidimensional plot such as a spider graph may also be included in a display of a patient monitor 900 as shown in FIG. 9. Patient monitor 900 may be a display of patient monitoring system such as patient monitoring system 104 of FIG. 1A. Patient monitor 900 may include spider graph section 902. Spider graph section 902 may include a spider graph 904 as well as a text box 906 including numbers corresponding to values shown on the spider graph. Spider graph section 902 may occupy a minority (e.g., less than 50%) of the display of patient monitor 900. In this way, patient monitor 900 may simultaneously include plot section 908 and vitals section 910. Plot section 908 may show plots of different physiological parameters over time. Vitals section 910 may show numerical indications of patient parameters. A clinician may wish to understand a current state of a patient by looking at all of the sections of patient monitor 900 at a single glance. By including a plurality of patient parameters in a shaped multidimensional plot, and the plot may be shown alongside plot section 908 and vitals section 910 such that all sections are readable by the clinician. Further, treatment recommendations may be shown in spider graph section 902 in the event that the patient becomes hypotensive.

The technical effect of methods 200, 300, and 400 is that a plurality of parameters may be automatically displayed at a display device of a patient monitoring system in a multidimensional graph. The multidimensional graph may enable a clinician to consider both current values and historical values as well as value thresholds in a single display area. Further, the clinician may be automatically alerted when the plurality of patient parameters is not being updated at the same rate and when one or more of the plurality of parameters varies beyond target ranges for the respective parameter. In the event that a patient becomes hypotensive, implementation of the methods may allow a timely administration of an effective treatment by an automatic treatment recommendation and in some examples, actuating an administration of the treatment. In some examples, the treatment may be automatically administered, thus decreasing an amount of time that the patient is in a hypotensive state.

The disclosure also provides support for a patient monitoring system, comprising: a display device configured to display a patient status view including one or more multidimensional plots overlaying more than one of real time values of a plurality of patient parameters, historical values of the plurality of patient parameters, and lower and upper threshold values of the plurality of patient parameters, and additionally being configured to display an abbreviated representation of at least one of the one or more multidimensional plots that can be reached directly from the patient status view, wherein the abbreviated representation displays a limited version of the respective multidimensional plot, the abbreviated representation being selectable to launch and enable the respective multidimensional plot to be seen, and wherein the abbreviated representation is displayed while in an unlaunched state. In a first example of the system, the one or more multidimensional plots further include text boxes with numbers corresponding to the real time values of the plurality of patient parameters, and wherein the abbreviated representation does not include the text boxes. In a second example of the system, optionally including the first example, the one or more multidimensional plots are automatically launched when at least one of the real time values of the plurality of patient parameters is outside of the lower and upper threshold values of a respective patient parameter of the plurality of patient parameters. In a third example of the system, optionally including one or both of the first and second examples, each of the one or more multidimensional plots corresponds to a different plurality of patient parameters. In a fourth example of the system, optionally including one or more or each of the first through third examples, the abbreviated representation includes upper and lower threshold values of a respective patient parameter of the plurality of patient parameters and/or a real time value of the respective patient parameter. In a fifth example of the system, optionally including one or more or each of the first through fourth examples, the one or more multidimensional plots and the abbreviated representation occupy less than 100% of an area of the display.

The disclosure also provides support for a method for monitoring a patient, comprising: obtaining, in real time, a plurality of patient parameters from monitoring devices coupled to the patient and storing the plurality of patient parameters on non-volatile memory of a computing unit, automatically calculating, with the computing unit, historical values of the plurality of patient parameters, and wherein the historical values of the plurality of patient parameters correspond to an average of stored values of the plurality of patient parameters obtained for a duration immediately preceding obtaining a real time value of the plurality of patient parameters, displaying, at a display device, a multidimensional plot including an overlay of real time values of the plurality of patient parameters, the historical values of the plurality of patient parameters, and upper and/or lower thresholds of the plurality of patient parameters, and indicating when at least one of the plurality of patient parameters is outside of the upper or lower threshold by generating a notification and displaying the notification at the display device. In a first example of the method, the method further comprises calculating, with the computing unit, a change in at least one of the plurality of patient parameters. In a second example of the method, optionally including the first example, the plurality of patient parameters includes blood pressure, an analgetic state parameter, a sedative state parameter, and a fluid management parameter. In a third example of the method, optionally including one or both of the first and second examples, the plurality of patient parameters further includes a peripheral perfusion parameter. In a fourth example of the method, optionally including one or more or each of the first through third examples, the notification includes a treatment recommendation based on the real time values of the plurality of patient parameters. In a fifth example of the method, optionally including one or more or each of the first through fourth examples, the treatment recommendation is automatically administered by an actuator coupled to the patient. In a sixth example of the method, optionally including one or more or each of the first through fifth examples, one of the plurality of patient parameters includes blood pressure and the treatment recommendation is in response to a real time value of blood pressure being below a threshold. In a seventh example of the method, optionally including one or more or each of the first through sixth examples, the change in at least one of the plurality of patient parameters is calculated by a difference between a real time value of one of the plurality of patient parameters and a corresponding historical value of one of the plurality of patient parameters. In a eighth example of the method, optionally including one or more or each of the first through seventh examples, the method further comprises: determining rates at which the plurality of patient parameters is obtained from each of the monitoring devices and generating an alert if the rates are not equivalent.

The disclosure also provides support for a system comprising: a display, monitoring devices, and a computer unit including non-volatile memory operably coupled to the display and storing instructions executable to: collect physiological signals in real time from the monitoring devices and convert the physiological signals to a plurality of patient parameters, store the plurality of patient parameters on the non-volatile memory, output, to the display, a shaped multidimensional plot including radial axes for each of the plurality of patient parameters and circumferential axes intersecting the radial axes according to numerical scales of each of the plurality of patient parameters, the radial and circumferential axes defining sections of the shaped multidimensional plot and the sections or portions of the sections being shaded according to a target range of the plurality of patient parameters, wherein the shaped multidimensional plot further includes at least one of a trace corresponding to real time values of the plurality of patient parameters and a trace corresponding to historical values of the plurality of patient parameters, and in response to the real time value of the plurality of patient parameters being outside of the target range, generate a notification and output the notification to the display. In a first example of the system, the shaped multidimensional plot further includes text boxes including numbers corresponding to real time values of the plurality of patient parameters and/or the historical values of the plurality of patient parameters. In a second example of the system, optionally including the first example, the notification includes a modification of a color or shading of a number and/or one or more sections of the shaped multidimensional plot corresponding to the one or more of the plurality of patient parameters outside of the target range. In a third example of the system, optionally including one or both of the first and second examples, the each of the historical values of the plurality of patient parameters are an average each of the plurality of patient parameters stored for a duration immediately prior to storing the real time value. In a fourth example of the system, optionally including one or more or each of the first through third examples, a shape of the shaped multidimensional plot is a circle or a polygon.

In another embodiment, the disclosure also provides support for a system, comprising: a display; and a computer unit operably coupled to the display and storing instructions executable to: output, to the display, a multidimensional plot including current values of a plurality of patient parameters, upper and lower thresholds of the plurality of patient parameters, and historical values of the plurality of patient parameters; and in response to one of the plurality patient parameters being outside of the upper or lower threshold values, generate a notification including a treatment recommendation and output the notification to the display.

In a further embodiment, the disclosure also provides support for a system comprising: a display, monitoring devices; and a computer unit including non-volatile memory operably coupled to the display and storing instructions executable to: collect physiological signals in real time from the monitoring devices and convert the physiological signals to a plurality of patient parameters store the plurality of patient parameters on the non-volatile memory output, to the display, the plurality of patient parameters as a multidimensional plot by overlaying one or more of real time values of the plurality of patient parameters, historical values of the plurality of patient parameters, and upper and lower threshold values of the plurality of patient parameters; and in response to the real time value of the plurality of parameters being outside of the upper or lower threshold value of the plurality of patient parameters, generate a notification and output the notification to the display, the notification including a treatment recommendation.

In an additional embodiment, the disclosure also provides support for A system, comprising: a display; and a computer unit operably coupled to the display and storing instructions executable to obtain blood pressure and patient parameters related to adequacy of anesthesia (AoA) and circulatory state from monitoring devices, calculate historical values and changes in blood pressure and the patient parameters related to AoA and circulatory state, output to the display a multidimensional plot including current values, historical values, and upper and lower threshold values of blood pressure and patient parameters related to AoA and circulatory state in response to blood pressure dropping below the lower threshold value, output a recommended treatment for raising blood pressure to the display.

Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A patient monitoring system, comprising:
a display device configured to display a patient status view including one or more multidimensional plots overlaying more than one of real time values of a plurality of patient parameters, historical values of the plurality of patient parameters, and lower and upper threshold values of the plurality of patient parameters, and additionally being configured to display an abbreviated representation of at least one of the one or more multidimensional plots that can be reached directly from the patient status view,
wherein the abbreviated representation displays a limited version of the respective multidimensional plot, the abbreviated representation being selectable to launch and enable the respective multidimensional plot to be seen, and
wherein the abbreviated representation is displayed while in an unlaunched state.

2. The patient monitoring system of claim 1, wherein the one or more multidimensional plots further include text boxes with numbers corresponding to the real time values of the plurality of patient parameters, and wherein the abbreviated representation does not include the text boxes.

3. The patient monitoring system of claim 1, wherein the one or more multidimensional plots are automatically launched in response to at least one of the real time values of the plurality of patient parameters being outside of the lower and upper threshold values of a respective patient parameter of the plurality of patient parameters.

4. The patient monitoring system of claim 1, wherein each of the one or more multidimensional plots corresponds to a different plurality of patient parameters.

5. The patient monitoring system of claim 1, wherein the abbreviated representation includes upper and lower threshold values of a respective patient parameter of the plurality of patient parameters and/or a real time value of the respective patient parameter.

6. The patient monitoring system of claim 1, wherein the one or more multidimensional plots overlay real time values of a plurality of patient parameters, historical values of the plurality of patient parameters, and lower and upper threshold values of the plurality of patient parameters.

7. A method for monitoring a patient, comprising:
obtaining, in real time, a plurality of patient parameters from monitoring devices coupled to the patient and storing the plurality of patient parameters on non-volatile memory of a computing unit;
automatically calculating, with the computing unit, historical values of the plurality of patient parameters, and wherein the historical values of the plurality of patient parameters correspond to an average of stored values of the plurality of patient parameters obtained for a duration immediately preceding obtaining a real time value of the plurality of patient parameters;
displaying, at a display device, a multidimensional plot including an overlay of real time values of the plurality of patient parameters, the historical values of the plurality of patient parameters, and upper and/or lower thresholds of the plurality of patient parameters; and
indicating that at least one of the plurality of patient parameters is outside of the upper or lower threshold by generating a notification and displaying the notification at the display device.

8. The method of claim 7, wherein the method further comprises calculating, with the computing unit, a change in at least one of the plurality of patient parameters.

9. The method of claim 7, wherein the plurality of patient parameters includes blood pressure, an analgetic state parameter, a sedative state parameter, and a fluid management parameter.

10. The method of claim 9, wherein the plurality of patient parameters further includes a peripheral perfusion parameter.

11. The method of claim 7, wherein the notification includes a treatment recommendation based on the real time values of the plurality of patient parameters.

12. The method of claim 11, wherein the treatment recommendation is automatically administered by an actuator coupled to the patient.

13. The method of claim 11, wherein one of the plurality of patient parameters includes blood pressure and the treatment recommendation is in response to a real time value of blood pressure being below a threshold.

14. The method of claim 8, wherein the change in at least one of the plurality of patient parameters is calculated by a difference between the real time value of one of the plurality of patient parameters and a corresponding historical value of one of the plurality of patient parameters.

15. The method of claim 7, further comprising determining rates at which the plurality of patient parameters is obtained from each of the monitoring devices and generating an alert if the rates are not equivalent.

16. A system comprising:
a display, monitoring devices; and
a computer unit including non-volatile memory operably coupled to the display and storing instructions executable to:
collect physiological signals in real time from the monitoring devices and convert the physiological signals to a plurality of patient parameters;
store the plurality of patient parameters on the non-volatile memory;
output, to the display, a shaped multidimensional plot including radial axes for each of the plurality of patient parameters and circumferential axes intersecting the radial axes according to numerical scales of each of the plurality of patient parameters, the radial and circumferential axes defining sections of the shaped multidimensional plot and the sections or portions of the sections being shaded according to a target range of the plurality of patient parameters, wherein the shaped multidimensional plot further includes at least one of a trace corresponding to real time values of the plurality of patient parameters and a trace corresponding to historical values of the plurality of patient parameters; and
in response to the real time value of the plurality of patient parameters being outside of the target range, generate a notification and output the notification to the display.

17. The system of claim 16, wherein the shaped multidimensional plot further includes text boxes including numbers corresponding to real time values of the plurality of patient parameters and/or the historical values of the plurality of patient parameters.

18. The system of claim 17, where the notification includes a modification of a color or shading of a number and/or one or more sections of the shaped multidimensional plot corresponding to the one or more of the plurality of patient parameters outside of the target range.

19. The system of claim 16, wherein the each of the historical values of the plurality of patient parameters are an average each of the plurality of patient parameters stored for a duration immediately prior to storing the real time value.

20. The system of claim 16, wherein a shape of the shaped multidimensional plot is a circle or a polygon.
